(19) United States
(12) Patent Application Publication
(10) Pub. No.: US 2006/0147425 A1
(43) Pub. Date: Jul. 6, 2006

(54) Use of enteric glia to promote functional nerve connections

(76) Inventors: Michel P Rathbone, Hamilton (CA); Shucui Jiang, Hamilton (CA); Jian Wang, Hamilton (CA); Pamela Middlemiss, Hamilton (CA); Mohammad Imtiaz Khan, Hamilton (CA)

Correspondence Address: KRAMER & AMADO, P.C., 1725 DUKE STREET, SUITE 240, ALEXANDRIA, VA, 22314, US

(21) Appl. No.: 10/531,425

(22) PCT Filed: Oct. 15, 2003
(86) PCT No.: PCT/CA03/01549

§ 371 Date: Mar. 3, 2006

Related U.S. Application Data
(60) Provisional application No. 60/418,167, filed on Oct. 15, 2002.

(51) Int. Cl. C12N 5/08; A61K 35/30

(52) U.S. Cl. 424/93.7; 435/368

(57) ABSTRACT

Methods of promoting functional regeneration of injured nerve fibers are described. The methods involve administering enteric glial cells to an animal with a nerve injury or neurodegenerative disease. The method is also useful in inducing formation of a blood-brain barrier in the nervous system.

### FIELD OF THE INVENTION

The invention relates to methods for promoting the functional regeneration of injured nerve fibers and methods for inducing the formation of a blood-brain barrier.

### BACKGROUND OF THE INVENTION

No treatment currently exists for central nervous system injuries, particularly for spinal cord injury (SCI). Following injury, peripheral nerves generally regenerate re-innervating their peripheral targets, and make largely appropriate reconnections (Fawcett and Keynes, 1990). However, injury to the central nervous system (CNS) particularly, the long tracts, axonal regeneration is largely abortive. This is largely attributed to the non-permissive nature of the glial environment in the CNS after injury (Bovolenta et al., 1992; Hatten et al., 1991; Horner, and Gage, 2000).

Several different approaches have been used in an attempt to overcome the inhibition of the nerve process outgrowth. Thus, attempts have been made to alter the immune responses of the myelin (Brittis and Flanagan, 2001; Chen et al, 2000; Prinjha et al, 2000). Another approach has been to add cells that can ‘chaperone’ the growing nerve fibers and shield them from the inhibitory effects of the myelin. Schwann cells, which form myelin in peripheral nerves, facilitate growth of nerve processes in the CNS after injury. However, Schwann cells tend to “wall themselves off”, thus making them a poor choice for regenerative cellular treatments (reviewed by Bartolomei and Greer, 2000). Other groups have used olfactory ensheathing glia (OEG) to promote regeneration (Pixley, 1992; Ramon-Cueto et al., 1994; 1995; 1998; Li et al., 1997; Navarro et al., 1999; Verdú et al., 2001). These unusual cells enhance outgrowth of olfactory nerve fibers under normal conditions (Barber, 1982) and “chaperone” regenerating nerve axons in the CNS (Doucette et al, 1983; Doucette, 1986).

The intestine is supplied by a very rich nervous system. It has many features similar to the CNS, such as a diversity of neuronal cell types, as well as other characteristic of the peripheral nervous system—e.g. its nerve cells can regenerate neuronal axons. The enteric nervous system and the olfactory nervous system have many similarities (Gershon, 1999). Enteric glia have properties similar to those of OEG, including axon-ensheathing properties, a lack of basal lamina and the presence of the unique glial protein, glial fibrillary acidic protein (GFAP) (Pixley, 1992; Gershon, 1999). Both EG and OEG show some of the properties of PNS Schwann cells and of CNS astrocytes (Pixley, 1992; Jessen, 1983; Gabella, 1981). But EG have several advantages over OEG as a source of cells fro spinal implantation: they are readily obtainable in very large quantities from the intestine without neurosurgery (Bernstein and Vidrich, 1994), and their migration in the spinal cord is not directional, a factor that limits the use of OEG (Ramon-Cueto et al., 1998; Wu et al., 1999). Moreover, EG can be easily obtained from an individual, cultured, and reinserted into the central nervous system of the same individual. Thus, potentially, EG can be used clinically in autologous transplantation.

### SUMMARY OF THE INVENTION

The present inventors have demonstrated that implantation of enteric glia (EG) into the spinal cord not only permits regeneration of injured nerve fibers into and through the spinal cord but also promotes these fibers to make functional connections.

Accordingly, the present invention provides a method of promoting the functional regeneration of injured nerve fibers in the nervous system comprising administering an effective amount of an enteric glial cell to an animal in need thereof. The present invention also provides a use of an effective amount of an enteric glia cell to promote the functional regeneration of injured nerve fibers in the nervous system. The invention further provides a use of an effective amount of an enteric glia cell for the manufacture of a medicament for promoting the functional regeneration of injured nerve fibers in the nervous system.

The inventors have also shown that transplantation of EG into the spinal cord can induce the formation of a blood-brain barrier (BBB). This is an important finding as the BBB can provide protection to the damaged nervous system from toxic substances.

Accordingly, the present invention also provides a method of inducing the formation of a blood-brain barrier in the nervous system comprising administering an effect amount of an enteric glial cell to an animal in need thereof. The present invention also provides a use of an effective amount of an enteric glia cell for inducing the formation of a blood-brain barrier in the nervous system. The invention further provides a use of an effective amount of an enteric glia cell for the manufacture of a medicament for inducing the formation of a blood-brain barrier in the nervous system.

The present invention further includes a pharmaceutical composition comprising an effective amount of enteric glial cells in admixture with a suitable diluent or carrier for use in promoting the functional regeneration of injured nerve fibers or for inducing the formation of a blood-brain barrier.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows GFAP and PHAL double-labeled EG cells in vitro.

FIG. 2 is a bar graph showing the proportion of Type II EG cells after 6 hours.

FIG. 3a shows a section of cord 7 days post EG transplantation. GFAP and PHAL double-labeled donor EG began to migrate into both white matter (dorsal column-DC) and gray matter (dorsal horn DH). 200X.

FIG. 3b shows a section of cord 1 month post EG transplantation. GFAP and PHAL double-labeled donor EG migrated at about 0.6 mm/day in both white matter (ventral column-VC) and gray matter (ventral horn-VH) into the ventral part of the cord. 200X.

FIG. 3c shows EM section of cord 1 month post EG transplantation. EG often in close contact either with neurons (a) or with axons (b, AX). Bars=1 μm (a) and 500 nm in (b).

FIG. 4a shows transplanted spinal cord after Evan's blue injection intravenously, a dye excluded by BBB.

FIG. 4b shows electron micrographs of capillary profiles in implanted spinal cord with EG (a) or medium (b) or with C6 cells after 2 months. EG implanted cord showed normal ultrastructure of capillaries and perivascular space (a). In contrast, control injected cord has now showed the formation of tight function (b and c). Bar=500 nm.

FIG. 5a shows the regenerating dorsal root axons entered the cord.

FIG. 5b shows EG enhance regeneration of axons and stimulate axons to penetrate through the CNS to their targets.

FIG. 6 is a bar graph showing the active area of the CTM reflex 1, 2 and 3 months after T13 DRC and transplantation with EG, 3T3, or medium at the dorsal root entry zone. Also shown (far right) is the area (means±SEM) of skin within the area innervated by mDCN-T13 that responded to a pinch by exhibiting a CTM reflex in unoperated controls.

FIG. 7 shows a micrograph of cross section from crush injured cord shows that the Hoechst-labeled EG stay at the injection site 1 day after transplantation. Scale bar=50 μm

FIG. 8 (a)-(b) shows micrographs of cross-section of spinal cords at the injection site after 7 days EG transplantation show the transplanted EG (blue) started to migrate from injection site, dorsal column (DC, white matter), to the gray matter (dorsal horn; DH). (a) Section from rat received EG implantation at 1 week after the initial crush injury. (b) Section from rat received EG transplantation at 5 weeks after the initial crush injury. Scale bar=55 μm for both.

FIG. 9 (a)-(d) show micrographs of horizontal sections of 1 month EG implanted cords at 1 week (a,c) and 5 weeks (b,d) after initial crush injury. (a) and (b) sections from 7.5 mm caudal from the injection center. (c) and (d) sections from 7.5 mm rostral from the injection center. Scale bar=55 μm.

### DETAILED DESCRIPTION OF THE INVENTION

I. Uses of EG

The inventors have shown that purified enteric glia (EG) from the small intestine are able, when transplanted into the dorsal root entry zone, to make functional connections within the spinal cord. The functional connections were evaluated by a behavioral test, the reflex activation of the cutaneous trunci muscle (CTM). The loss of CTM reflex after injury to the spinal cord (the sensory fibers comes from dorsal root) is observed as a region of dorsal skin, which fails to respond by muscular contraction to local tactile stimulation. The inventors have found that EG-implanted animals had a statistically significant recovery of the CTM reflex. This indicates, that EG not only permits regeneration of injured nerve fibers into the spinal cord, but also promote these fibers to make functional connections.

Accordingly, the present invention provides a method of promoting the functional regeneration of injured nerve fibers in the nervous system comprising administering an effective amount of an enteric glial cell to an animal in need thereof. The present invention also provides a use of an effective amount of an enteric glia cell to promote the functional regeneration of injured nerve fibers in the nervous system. The invention further provides a use of an effective amount of an enteric glia cell for the manufacture of a medicament for promoting the functional regeneration of injured nerve fibers in the nervous system.

The term “enteric glial cell” as used herein means a glial cell obtained from the enteric nervous system. Preferably, the EG cell is a Type II EG cell that has many long processes and has high levels of glial fibrillary acidic protein (GFAP). Most preferably, prior to transplantation, the EG cells are purified and cultured in vitro. In a preferred embodiment, the EG cells are obtained from the animal to be treated, purified and cultured in vitro and then re-inserted into the same individual as an autologous transplantation.

The term “a cell” as used herein includes a single cell as well as a plurality or population of cells.

The term “effective amount” as used herein means an amount effective at doses and for periods of time necessary to achieve the desired amount, e.g. to promote functional regeneration of injured nerve fibers.

The term “promoting the functional regeneration of injured nerve fibers” as used herein means that the functional connection between the nerve fibers in the presence of the EG cells is greater than observed in the absence of the EG cells. The presence of a functional connection between the nerve fibers can be tested using techniques known in the art. In one embodiment, the functional connections can be evaluated by a behavioral test such as the reflex activation of the cutaneous trunci muscle (CTM) as described in Example 1.

The term “nervous system” as used herein includes both the peripheral nervous system (PNS) and the central nervous system (CNS).

The term “animal” as used herein includes all members of the animal kingdom, including humans. Preferably, the animal to be treated is a human having a condition requiring nerve regeneration. Most preferably, the person has a CNS or PNS injury or a neurodegenerative disease. Examples of neurodegenerative diseases that may be treated according to the present invention include Alzheimer's disease, Parkinson's disease, multiple sclerosis, Huntington's disease, Bell's palsy, Pick's disease and amyotrophic lateral sclerosis. Examples of nerve injuries include neurotrauma, stroke and cerebral ischemia as well as peripheral nerve injuries or neuropathies of any type, including traction injuries, paralysis and neuropathic (neurogenic) pain syndromes.

As previously mentioned, the inventors have also shown that transplantation of EG into spinal cord can also induce the formation of the blood-brain barrier (BBB). Accordingly, the present invention also provides a method of inducing the formation of a blood-brain barrier in the nervous system comprising administering an effect amount of an enteric glial cell to an animal in need thereof. The present invention also provides a use of an effective amount of an enteric glia cell for inducing the formation of a blood-brain barrier in the nervous system. The invention further provides a use of an effective amount of an enteric glia cell for the manufacture of a medicament for inducing the formation of a blood-brain barrier in the nervous system.

II. Compositions

The present invention also includes pharmaceutical compositions comprising enteric glial cells for use in the methods of the invention. Accordingly, the present invention includes a pharmaceutical composition for promoting the functional regeneration of injured nerve fibers comprising an effective amount of enteric glial cells in admixture with a suitable diluent or carrier.

The present invention also includes a pharmaceutical composition for inducing the formation of a blood-brain barrier comprising an effective amount of an enteric glial cell in admixture with a suitable diluent or carrier.

Such pharmaceutical compositions can be administered in any known manner to deliver the cells to the nervous system including intralesional, parenteral, subcutaneous, intrathecal, transperitoneal, and intracerebral modes.

The pharmaceutical compositions of the invention can be intended for administration to humans or animals. Dosages to be administered depend on individual needs, on the desired effect and on the chosen route of administration.

The pharmaceutical compositions can be prepared by per se known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

On this basis, the pharmaceutical compositions include, albeit not exclusively, the EG cells in association with one or more pharmaceutically acceptable vehicles or diluents, and contained in buffered solutions with a suitable pH and iso-osmotic with the physiological fluids. The pharmaceutical compositions may additionally contain other agents such as other agents that can be used to treat nerve injuries or neurodegenerative diseases. Such agents include purine nucleosides such as guanosine and inosine.

The following non-limiting examples are illustrative of the present invention:

### EXAMPLES

### Example 1

(i) Method for Isolation and Purification of EG from Rat Small Intestine

Purified enteric glia must be isolated from the myenteric plexus in significant numbers, free from other cell types, for transplantation into the damaged spinal cords. Previous methods isolation of enteric glia from the myenteric plexus of various species involved the explant techniques (Ruhl et al., 2001; Bannerman et al., 1988) and/or the use of antibodies and complement (Ruhl et al., 2001; Bannerman et al., 1988) to achieve antibody complement—mediated cytolysis, to remove the contaminating neurons and fibroblasts. Other methods used supplementation of EG cultures with expensive factors, such as fibroblast growth factor, brain pituitary extract and forskolin (Saffrey, 1984; Ruhl et al., 2001) to promote growth of the isolated enteric glia. The inventors have modified the isolation method of (Bernstein, 1994) and included a step to removes contaminating cells, and a second step to separate isolated enteric glia from the feeder layer of growth arrested Swiss mouse 3T3 cells (Middlemiss et al., 2002, In Vitro, Cell Dev Biol-Animal).

(ii) There are Two Morphologically Distinct EG Types

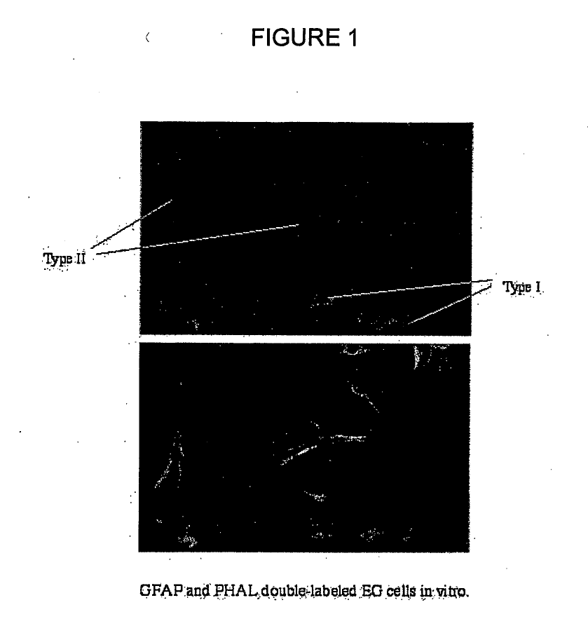

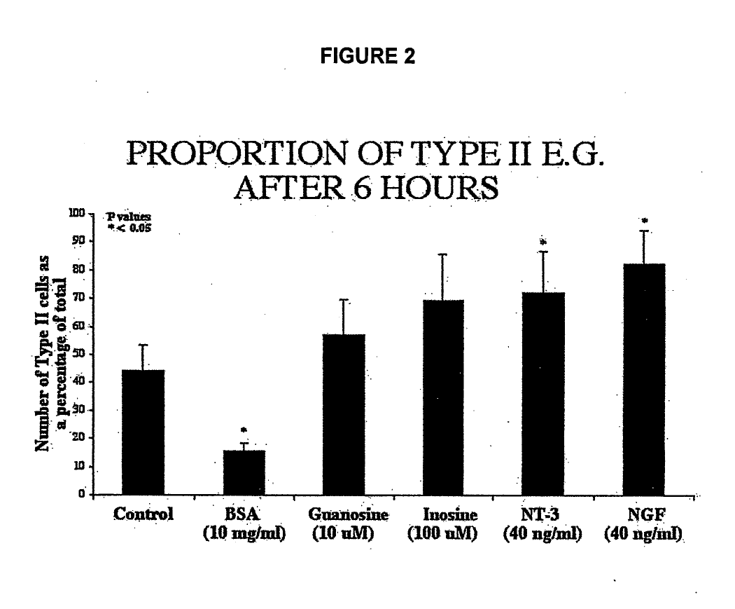

It was known, that in the central nervous system, astrocytes exist in two distinct sub-types, one of which is a better substrate for neuronal survival and neurite outgrowth than the other. The inventors found, that the EG consist of two morphologically-distinct cell types. One type (Type I) has very few processes and contains little glial fibrillary acidic protein (GFAP). The other type of cell, (Type II), has many long processes and has high levels of GFAP (FIG. 1). Moreover, the inventors showed that these cells can inter-convert over time, and that guanosine and inosine, as well as neurotrophic factors such as NGF and NT-3, enhance this interconversion (FIG. 2).

(iii) EG Survive and Migrate after Transplantation into the Spinal Cord

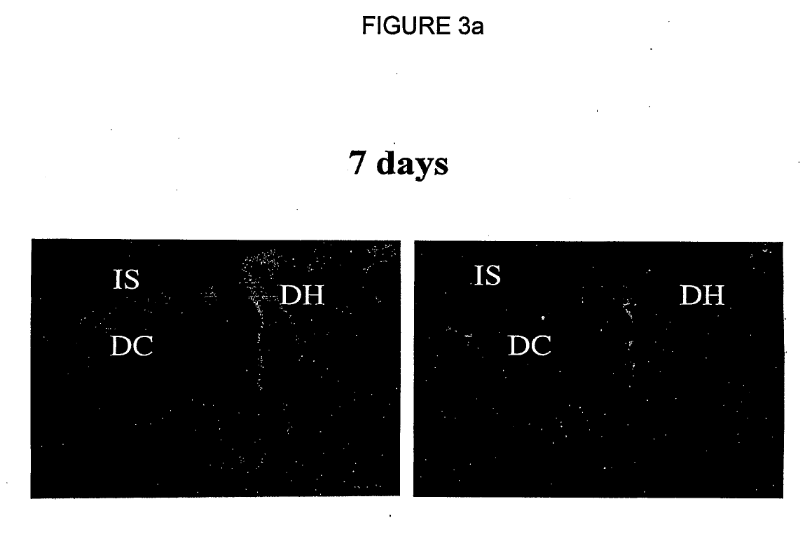

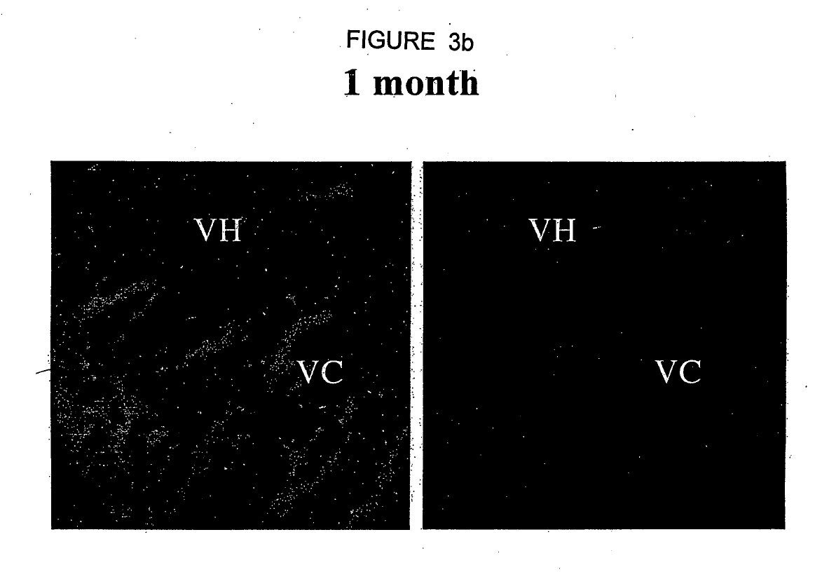

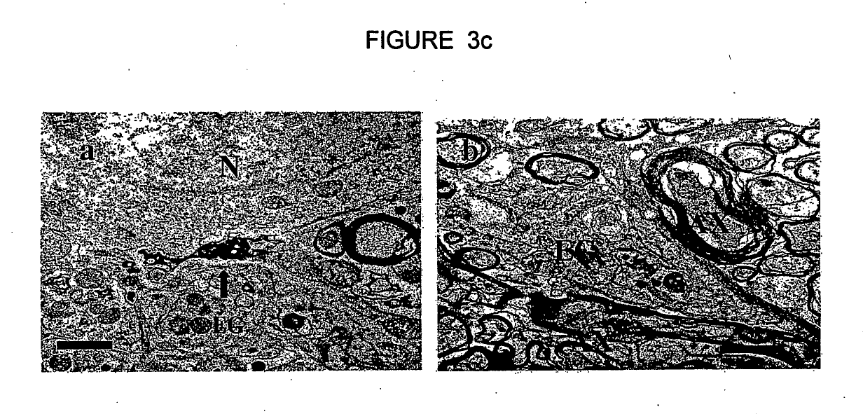

Light Microscopic Analysis: The ability of EG to survive, and migrate after transplantation into the CNS, is paramount if they are to be used as a “chaperone” for regenerating nerve fibers. The inventors have found that after implantation into spinal cord EG survived and migrated for up to 3 months (FIG. 3b), starting at 7 days (FIG. 3a). EG migrated through the spinal cord at ˜0.6 mm/day during the first month and at a rate of ˜0.4 to 0.5 mm/day for the second and third months. The cells most distant from the implantation site migrated at least 3.5 to 4 cm over the term of the experiment.

Only Type II EG cells, which are fibrous cells with long, thin processes migrated from the implantation site, and survived for the duration of the study (FIGS. 3a and b). Most of the cells were identified as Type II immediately prior to transplantation. However, some Type I cells were also present. The presence of only Type II cells in the migrating cell population suggests, that following transplantation Type I EG inter-converted into Type II EG, or they died after transplantation into the spinal cord.

Ultrastructure of EG and Interrelationship with Host Spinal Tissues: Immuno-staining with diaminobenzidine (DAB) to visualize PHAL-labeled EG showed, that the cytological features of EG. These were flattened, exhibited dense packing with intermediate filaments, or gliofilaments (a characteristic of enteric glia). EG and host cells were intermingled. Importantly, EG sent out extensive processes into the host neuropil, where they partially ensheathed the axonal fibers. EG were often in close contact with the neuron or neuronal processes (FIG. 3c).

(iv) EG Induce the Formation of the Blood-Brain Barrier (BBB)

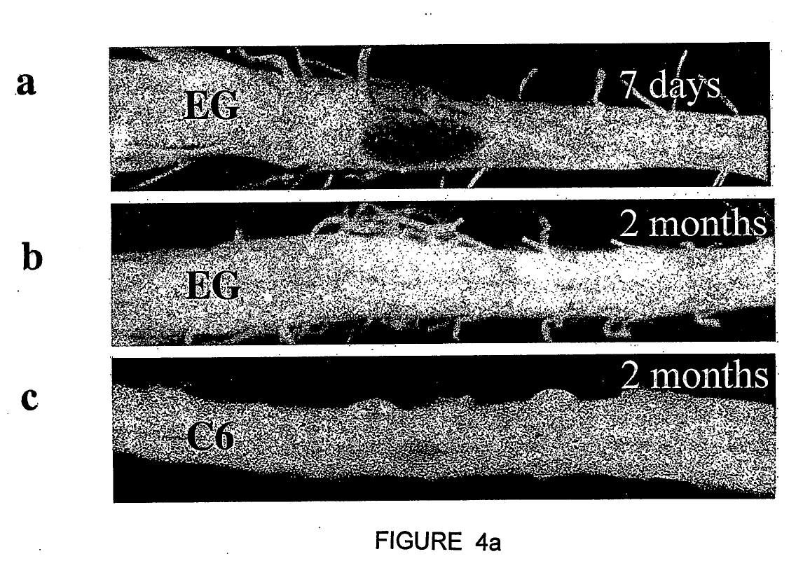

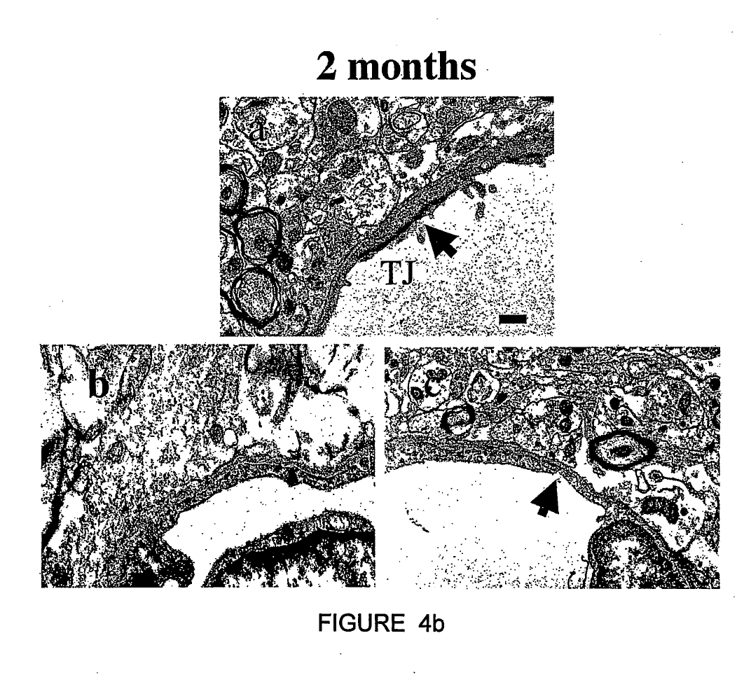

A type of CNS glia, known as astroglia, which have some similarities to EG, are known to induce the formation of blood capillaries in the nervous system an important part of the blood-brain barrier (BBB). This ‘barrier’ excludes many potentially toxic substances from entering the brain and the spinal cord from the blood and the extracellular fluid. Therefore, it is important to demonstrate whether enteric glia induce BBB characteristics in the small blood vessels that vascularize grafts of EG after transplantation into the spinal cord. The inventors have found, that when EG are transplanted into the spinal, cord no barrier is detected in the vascularizing capillaries up to one month after surgery. In contrast, after 2 and 3 months following EG implantation a barrier appears to have formed (FIGS. 4a and b). This is the first demonstration, that transplanted EG in the spinal cord can induce the formation of a BBB. This barrier, formed following EG transplantation will provide protection to the CNS from many potentially toxic substances.

(v) EG Permit Regeneration of Cut Dorsal Root Axons into the Spinal Cord

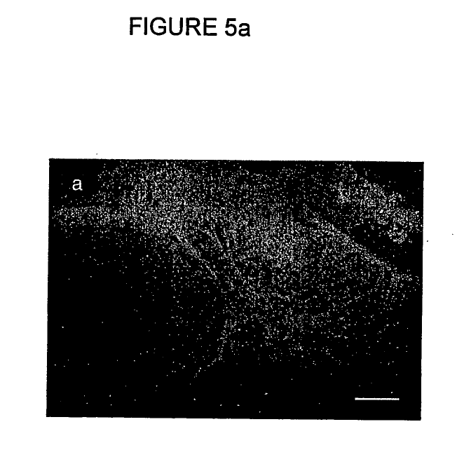

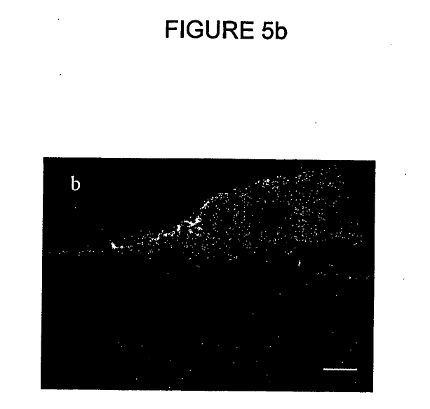

We transected the T13 dorsal root of rats at its entry zone into the spinal cord. Normally, the central branch of the dorsal root ganglion neurons regenerate through the Schwann cellsconyained in the root, but do not enter the CNS, their further growth being apparently inhibited by CNS myelin. However, when EG were implanted at the dorsal root entry zone, the regenerating dorsal root axons entered the cord (FIG. 5a), whereas they did not do so in the control animals (FIG. 5b). This indicates, that EG enhance regeneration of axons and stimulate axons to penetrate through the CNS to their targets.

(vi) EG-Induced Regenerating Axons Make Functional Connections

We evaluated the extent of functional regeneration of cut dorsal root fibers into the spinal cord following EG transplantation, by using a behavioral test. The inventors assessed the reflex activation of the cutaneous trunci muscle (CTM) by the nociceptive fibers of the dorsal cutaneous nerves (DCNs). The CTM is a vast sheet of muscles attached to the underside of the skin of the back and the flanks, and each segmental DCN activates a functionally appropriate area of that muscle, which extends below and somewhat beyond the sensory dematome. The loss of CTM reflex after injury to the spinal cord (the sensory fibers emerge from dorsal root) is observed as a region of back skin, which fails to respond by muscular contraction to local tactile stimulation. Normally, after T13 dorsal root cut, the relevant (DCN-T13) sensory field of areflexia does not recover for the life of the animal.

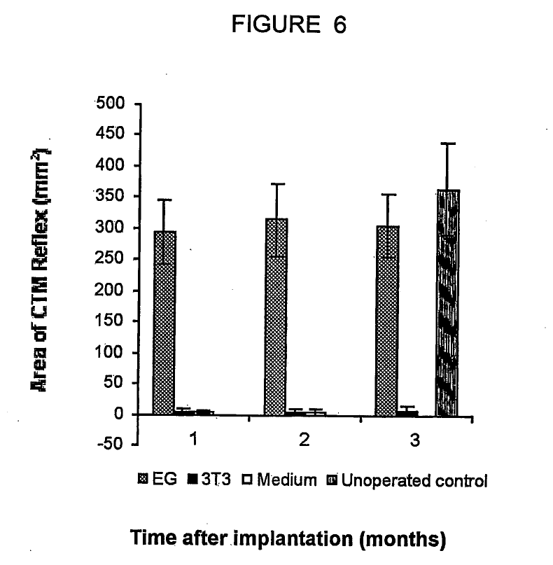

In present study, the regeneration of T13 dorsal root fibers was studied in dorsal skin containing an “isolated” nerve field (mDCN-T13) on the right side of the midline. Because adjacent nerve fields have been denervated (T10, T12 and L1-L3), the response area (mean±EM) of the CTM reflex pinch by forceps indicates the recovery of mDCN-T13 after T13 dorsal root cut (DRC). The results show, that in EG-implanted animals the inventors found a statistically significant recovery of the CTM reflex (FIG. 6). This indicates, that the implantation of EG into the spinal cord not only permits regeneration of injured nerve fibers into and through the spinal cord, but also promotes these fibers to make functional connections.

Materials and Methods

(i) Isolation and Purification of EG from Rat Small Intestine

Adult female (200-250 gm) Wistar rats (Charles River, Montreal Canada) were euthanized, and the small intestine was removed. The small intestine was then placed in a sterile petri dish (Fisher-Toronto, Canada), into cold phosphate buffered saline (PBS, pH 7.6), containing antibiotics and fungizone (gentamycin (100 μg/mL, penicillin 100U/mL, streptomycin 100 μg/mL and fungizone 250 μg/mL (Life Technologies, Grand Island, N.Y.). The intestine was cut into 1 cm segments and the segments were cut open and washed 6 times for 10 minutes with PBS containing antibiotics and fungizone (as described above). The segments were then treated for one hr with 3-6 U/mL dispase (Life Technologies, Grand Island, N.Y.) in Dulbecco's modified Eagle medium (DMEM, Life Technologies, Grand Island, N.Y.) containing 10% fetal calf serum (FCS-Life Technologies, Grand Island, N.Y.), penicillin (100U/mL), streptomycin (100 μg/mL) and gentamycin (50 μg/ml) (Medium A) at 37° C. in a humidified CO₂ incubator with manual agitation every 15 min. After 1 hour the serosal and mucosal surfaces were gently scraped with a scalpel blade, and the cells recovered by centrifugation at 800 g for 4 min. The remaining adhering mucosa was dissolved by treatment with 1% N-acetylcysteine (ICN, Costa Meta, Calif.) at room temperature for 15 min. Cells recovered by centrifugation (800 g, 4 min) were washed once with medium A, and resuspended in DMEM supplemented with 20% FCS, penicillin (100U/mL), streptomycin (100 μg/mL), insulin (5 μg/mL-Life Technologies, Grand Island, N.Y.), transferrin (5 μg/mL-Sigma, Oakville, Canada), sodium selenite (5 ng/mL-Sigma, Oakville, Canada), and hydrocortisone (0.5 μg/mL-Sigma, Oakville, Canada), and then were filtered through a nylon mesh with openings of 74 μm (Small Parts Inc, Miami Lake, Fla.). Isolated cells were plated at 1×10⁴ cells/mm² onto rat-tail collagen-coated wells of a 6 well tissue culture plate (NUNC, Naperville, Ill.). Tissue culture inserts (NUNC, Naperville, Ill.) were also coated with rat-tail collagen and were seeded with a feeder layer of growth-arrested 3T3 Swiss albino mouse embryo fibroblasts (3T3 cells-ATCC, Rockville, Md.) (Bernstein, 1994). One insert was added to each well of the plate to provide the necessary growth factors. The medium was changed 4 days after plating, and every 3 days thereafter. After the third change of medium the serum concentration was lowered to 10% and cholera toxin (250 ng/ml-Calbiochem, Homby, Ontario) was added to the wells to promote growth of enteric glia. Purified glia (1×10⁴ cells/mm²) were then seeded onto 12 mm round coverslips, coated with poly-L-lysine for staining purposes or were labelled with PHAL for transplantation.

(ii) In vitro Histological Studies

Isolated EG were stained for glial fibrillary acidic protein (GFAP) the standard marker for astroglia using immunocytochemical techniques (Beltz, 1989). The three-step Streptavidin-Biotin-Peroxidase method was used to demonstrate the presence of GFAP in these cells. Briefly, the method is as follows: fixed and permeabilized cells were pretreated with normal swine serum to block the non-specific binding sites in the cell, then incubated for 2 hours with unlabeled primary antibody (anti rabbit cow GFAP, 1:500, (Dako), and rinsed with 0.1M phosphate buffer solution. The cells were then incubated for one hour with the secondary antibody (biotinylated goat anti-rabbit IgG. 1:100, (Dako), rinsed with 0.1M phosphate buffer, and then incubated with streptavidin-horse radish peroxidase for 1 hour (1:500), (Dako). Cells were then rinsed with 0.1M phosphate buffer, treated with 3,3-diaminobenzidine tetrahydrochloride (DAB) and hydrogen peroxide for 2-5 minutes, then again rinsed with 0.1M phosphate buffer. Coverslips were placed onto the slides, and then viewed for the presence of GFAP. Over 98% of the cells stained positively for GFAP (FIG. 1).

(iii) Transplanted EG Induce Blood-Brain Barrier (BBB) Formation

In this study, the inventors assessed the properties of the BBB, following EG transplantation into the spinal cords in Evan's blue died tissues, using electron microscopy (EM).

Cell preparation and Transplantation: Cultured enteric glia was treated with 2 μg/mL antibody to Phaseolus vulgaris leuco-Agglutinin (PHA-L) for 1 hour at 37° C. After thorough rinsing with phosphate buffered saline (PBS), the PHA-L-labelled cells were aspirated into a 26 gauge needle, attached to a 5 μl Hamilton syringe.

Following laminectomy of deeply anesthetized (pentobarbital, 65 mg/kg body weight) adult host (300 g) Wistar female rats, 2 μl of PHAL-treated EG cell suspension (concentration: 1×10⁴/μl) was pressure injected, subdurally, on the left side between the dorsal horn and column at the level of the eleventh thoracic vertebrae (T11). Other rats were injected with the same amount and the same concentration of either fibroblast (3T3), or C6 glioma cells to serve as negative controls. Yet other rats were injected with 2 μl of either PBS solution or culture medium to serve as vehicle controls. After implantation, the wounds were closed. Sham-operated control rats were similarly injected at T11 either with PBS solution or with culture medium without any cells. This implantation technique did not cause any loss of mobility or any bladder or defecation problems in the experimental animals.

After 1 to 3 months, the implanted rats were anesthetized with sodium pentobarbital (65 mg/kg, 0.1 ml/kg) and then injected intravenously over 2 min via a tail vein with Evan's Blue dye (2%, 5 ml/kg body weight). The whole body of the rats turned blue immediately after injection. 20-30 min after the injection of the Evan's blue dye rats were perfused with saline, and the spinal cords were removed from each rat, examined and photographed macroscopically before sectioning with a vibratome.

Consecutive vibratome sections (100 μm) were cut in transverse planes from experimental and control animals. One, 2, 3 weeks, and 1, 2, 3 months post-implantation several slices were selected from the site at which the cells were injected. In order to visualize PHAL labelled EG cells within the spinal cord, the following procedure was carried out. First, floating sections were rinsed thoroughly with PBS. After several steps of reducing background staining, including incubation with 0.3% H₂O₂ in absolute methanol, Tris glycine buffer, 1% sodium borohydrite and serum blocking solution (PBS containing 5% Donkey serum), sections were incubated overnight at 4° C. with primary antibody (goat anti-PHAL, Vector Labs) diluted to 1:500 in PBS containing 0.25% Triton X-100, 1% bovine serum albumin. For reagent controls the primary antibody was replaced with 5% Donkey serum. Sections were then incubated with the secondary anti body (Biotinylated anti goat IgG) for 30 minutes. After washing with PBS, sections were incubated for 5 minutes with VECTASTAIN Elite ABC Reagent and then washed with PBS. Finally, sections were incubated for 2-5 minutes with DAB substrate kit for peroxidase (Vector Laboratories) and washed 2 times with PBS and 3 times with 0.1M cacodylate buffer. Finally, sections were post-fixed with 2% osmium tetroxide (OsO4) for one hour, dehydrated in a graded series of ethanol, and embedded in epoxy resin. Then semi-sections (1 μm) were cut and stained with 1% Toluidine blue. Areas, which showed the best structural integrity in dorsal central portions of the white and grey matter of the injected segment were then selected from each slice to be used for thin sectioning. Ultrathin sections were cut on a ultramicrotome (Reichert Ultracut S, Leica, Vienna, Austria) at 70 nm, stained with uranyl acetate and lead nitrate, and examined with an JEOL 1200 electron microscope.

(iv) EG Survive and Migrate after Transplantation into the Spinal Cord

The basic procedures were the same as described above. The inventors used different animals, which, unlike those in the BBB studies, were not injected with Evan's blue prior to further processing. A brief outline of the procedure is presented below:

Cell preparation and Transplantation: Enteric glia were prepared from the rat intestine and grown in vitro as described above. 2 μl of PHAL-treated EG suspension (concentration: 1×10⁴ cells/μl) were injected into the left side of the rat, between the dorsal horn and the column at T11 to T12 level. Separate rats were injected with the same amount and same concentration of either fibroblast suspension (3T3) or C6 glioma cells, and these served as negative controls. Different rats were also injected with 2 μl of either phosphate buffered saline (PBS) solution, or culture medium, and these served as vehicle controls.

Fluorescent light microscopy: After perfusion, cords were removed as described above. Then, cords were cryo-protected in 30% sucrose solution and frozen at −70° C. in 10.24% polyvinyl alcohol and 4.26% polyethylene glycol embedding medium. Cords were sectioned into 18-25 μm transverse sections at 10 mm to the crush site, both in anterior, and caudal direction. Every section was examined, and every fifth section was stained with GFAP and PHAL double fluorescence to detect the implanted EG cells. Briefly, the sections were incubated overnight with the primary antibody and combined antisera to GFAP (rabbit anti-GFAP) and PHAL (goat anti-PHAL) diluted (1:600 each) in PBS, containing lambda carrageenank, Triton X-100 and donkey serum. They were then incubated with combined donkey antisera to goat IgG (fluorescein [FITC]-conjugated, Green) and donkey antisera to rabbit IgG (rhodamine [RITC]-conjugated, Red). FITC fluorescence was enhanced by goat anti mouse IgG and mouse anti-goat IgG.

Electron microscopy: One, two or three months after transplantation, animals were perfused, the spinal cord removed and consecutive vibratome sections were cut in transverse planes from both, control and experimental animals. Immunostaining was carried out to visualize PHAL labeled EG cells within the spinal cord. Briefly, after several steps of reducing the background staining, sections were incubated overnight at 4° C. with the primary antibody. In reagent controls the primary antibody was replaced with serum. After 16 hours, sections were incubated with the secondary antibody, then incubated with ABC (avidin-biotin complex) reagent. After washing with PBS and with 0.1M cacodylate buffer, the sections were postfixed with osmium tetroxide (OsO4) for one hour. After dehydration in a graded series of ethanol, these were embedded in epoxy resin, and semi-section (1 μm) were cut and stained. After the best-preserved areas were selected, ultra thin sections were cut on an ultramicrotome, stained with uranyl acetate and lead nitrate, and examined with an JEOL 1200 electron microscope.

(v) Transplanted EG Permit Regeneration of Cut Dorsal Root Axons into the Spinal Cord

We used the experimental design described by Ramon-Cueto and Nieto-Sampedro (1994). Essentially, under ketamine/xylazine anesthesia of young female rats, a laminectomy was performed at T11, and the right dorsal root and cord were exposed. The dorsal root was cut at its entry point into the spinal cord and reanastomosed to its anatomical entry point into the spinal cord using 10-0 suture silk. Enteric glia (1×10⁵ cells/mL culture medium) was drawn into a siliconized glass pipette (100 μm O.D., 80 μm I.D.) attached to a Hamilton syringe (5 μL). 2 μL of cell solution was injected at 1 μL/minute into the spinal cord 0.2 mm from the dorsal surface at the point of anastomosis of the dorsal root. Control animals received the same amount and at the same concentration of either fibroblast (3T3), or glioma (C6) cells, or only fresh EG-culture medium. Further controls received only surgery to the root without injection.

Three weeks after surgery animals were deeply anesthetized and perfused transcardially with 4% paraformaldehyde in 0.1M phosphate buffer, pH 7.3. The spinal cords were removed and the anastomosed root cut from the spinal cord. In spinal cords in which the regeneration of the dorsal root was examined, a crystal of 1,1′dioctadecyl-3,3,3′,3′-tetramethylindocarbocyanine perchlorate (DiI) was placed at the cut stump of the nerve root to aid this process.

(vi) EG-promoted Regenerating Fibers Make Functional Connections

The reflex activation of the cutaneous trunci muscle (CTM; Theriault and Diamond, 1988a, b) by the nociceptive fibers of the dorsal cutaneous nerves (DCNs), was used to evaluate the functional regeneration of cut dorsal root fibers into the spinal cord following EG transplantation. The CTM is a vast sheet of muscle attached to the underside of the skin of the back and flanks, and each segmental DCN activates a functionally appropriate area of that muscle which extends below and somewhat beyond the sensory dematome. The loss of CTM reflex after injury to the spinal cord (the sensory fibers arise from the dorsal root) is observed as a region of the skin on the back, which no longer responds by muscular contraction to local tactile stimulation. Normally, after T13 dorsal root cut, the relevant ((DCN-T13) sensory field of areflexia, does not recover for the life of the animal.

Surgical “isolation” of cutaneous sensory fields: One month after dorsal root reanastomosis and cell transplantation (as described above) the sensory field of T13 dorsal cutaneous nerve (DCN-T13) was “isolated” on the right side of the rat using the technique described by Pertens et al. (1999). This procedure involves the surgical elimination of a number of the neighboring thoracic and lumbar DCNs (T10-T12 and L1-L3, and the lateral branch of DCN-T13), and the lateral cutaneous nerves T10-L3, as described by Doucette and Diamond (1987). This results in a selected nerve field, totally surrounded by a large expanse of denervated skin. The inventors no longer find it necessary to remove DCNs on the left side of the animal; the longitudinal skin incision routinely made on the left of the midline does cut distal branches of the left DCNs, but the inventors have now confirmed that, if these regenerate, then they never cross the incision line to reach the skin to the right of the incision; thus, this skin can become reinnervated only by axons originating in DCNs of the right side.

Behavioral testing—behavioral mapping of nociceptive fields: The mapping techniques were those described by Nixon et al. (1984) and Doucette and Diamond (1987). Briefly, 3 days after surgical “isolation” of the cutaneous sensory fields, under light sodium pentobarbital anesthesia (30-35 mg/kg, i.p.), mechanonociceptive (“pinch”) fields were mapped by systematic application across the back skin of pinching with specially ground down, fine-toothed forceps. The presence of nociceptive endings was indicated by the characteristic “CTM reflex response”, a brisk puckering of the skin due to the activation of the underlying CTM by the impulses generated in DCN nociceptive axons. The border between denervated (and, thus, reflexly inactive) and innervated skin was marked with a fine fiber-tipped pen, and the mapped areas were traced onto transparent acetate sheets for measurement with an image analysis system. The data were analyzed using analysis of variance (ANOVAs).

Discussion

Transplantation of pure EG provides the first evidence that functional repair of damaged adult sensory axons can be achieved. The inventors have demonstrated functional evidence for the activation of sensory neuron targets and the recovery of reflexes with associated behavioral functions. These studies show that transected PNS afferents could cross the PNS—CNS boundary and re-grow into the adult spinal cord with the assistance of EG transplants. The mechanisms by which EG promote sensory fiber spinal ingrowth and functional recovery are not known, but a combination of the unique properties of EG likely mediates the axonal regeneration. EG may elaborate trophic factors, which strongly promote axonal growth (Tew et al., 1994). Alternatively, they may protect axons from the inhibitory influence of the CNS myelin (Brittis and Glanagan, 2001, Schwab, 2000) either by producing a facilitatory extracellular matrix, or by physically separating the tips of the growing axons from the host myelin. The ability of EG to ensheath axons and reduce the toxic substances entering the central nervous system from the blood by inducing BBB formation may also be important. EG may also migrate to the PNS—CNS boundary, and ensheath a bundle of axons, isolating them from the inhibitory reactive astroglial environment.

### Example 2

Enteric glial cells survive and migrate after transplantation into injured (crush) spinal cord

As described above in Example #1 that EG survived and migrated after they were transplanted into normal spinal cord and spinal cord following dorsal root cut. In the present study the inventors have transplanted EG into injured spinal cord (crush model see below). The present data lead to conclude that the implanted EG survived, migrated and remained “competent” to promote the regeneration of spinal cord axons after delayed transplanted into the injured area.

Materials and Methods

Enteric glia were prepared from the rat intestine and grown in culture as described previously.

Adult female Wistar rats (280-300 g weight, Charles River) were anaesthetized with isoflurane (3-5%): O2 (1L/min). Buprenorphine (0.03 mg/kg body weight, subcutaneously) was administered prior to surgery for pain relief. Spinal cords were surgically exposed and crushed with modified coverslip forceps (Blight, 1991; Gruner et al., 1996) producing a moderate spinal cord injury Briefly, the backs of anaesthetized animals were shaved and cleaned with providing and alcohol. The vertebrae were exposed and a dorsal laminectomy performed at T12 to expose the cord, leaving the dura intact. The forceps were closed slowly (over 2 seconds) over the entire width of the spinal cord, to a thickness of 1.4 mm, held closed for 15 seconds and then removed. Suturing the vertebral muscles closed wounds and fat pad over the wound and closing the skin with wound clips. Postoperatively, rats were kept quiet and warm.

EG suspension, prelabelled with bisbenzimide, a nuclear fluorochrome (Hoechst 33342) were prepared immediately before implantation. Some rats were injected immediately following the initial crush injury. The others were re-operated at the initial injury site at 1 or 5 weeks following spinal cord crush injury. Either a total of 2×10⁶ cells/μl EG or 2 μl medium only were injected into the crush area.

The survival of EG in the injured spinal cord was monitored by sacrificing the animals at 1 day or 1 week, or 1, or 3 months after implantation. Animals were perfused the T9-L3 segment, which included the crushed injury, were removed and embedded. Serial sections were cut for the histological processing and identifying the surviving and migration of Hoechst-positive EG in the sectioned spinal cords using fluorescent light microscopy. The injured center of each cord was cross-sectioned for observing the location of injected site and for localizing the labeled EG cells in the dorsal, ventral and lateral matter of the cord. Longitudinal sections were cut for determining the migrating distances, particularly, in the rostral and caudal directions from the center of the injury:

Results

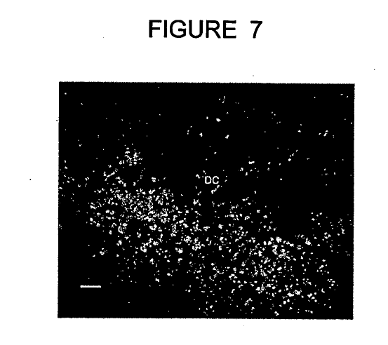

1 DPI (day post-injury): Transplanted Hoechst-positive EG were confined to a cyst in the dorsal column of host cord, the transplantation needle tract, or the dorsal surface (FIG. 7). No obvious migration could be seen. The EG cells survived well in both immediately and delayed (1 or 5 weeks) implanted cords of animals.

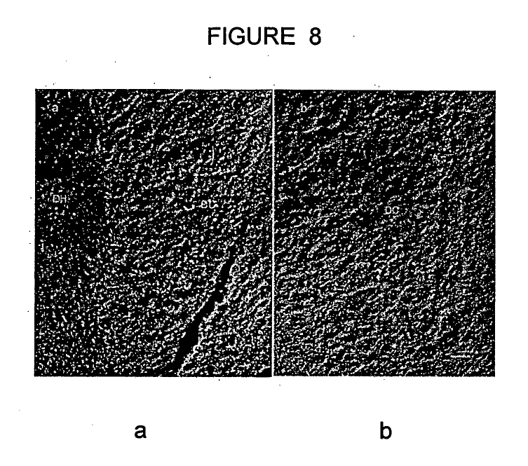

7 DPI: Donor EG, identified by Hoechst (blue) were observed within a limited area close to the transplant but also started to the gray matter of the host (FIG. 8a,b). The distances EG migrated in the different groups, i.e. immediately and delayed implanted cords were the same.

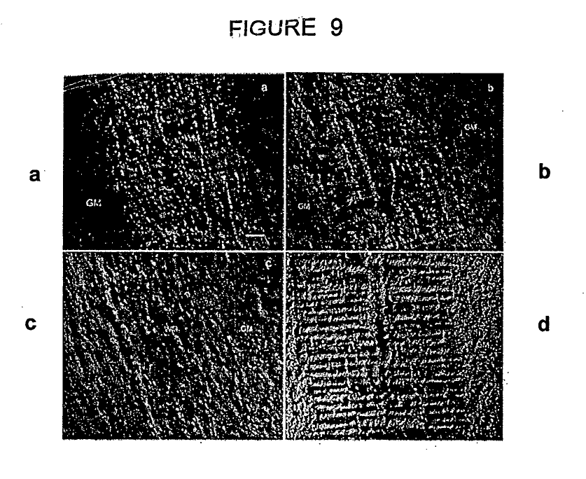

1 MPI (month post-injury): EG cells have migrated longer distances from the injected site in both gray and white matter. Cells crossed the injured area and migrated laterally into dorsal horn and then proceeded in a caudal (FIGS. 9a and b) and rostral direction (FIGS. 9c and d).

### CONCLUSIONS

The inventors conclude that, unlike the cells implanted one week or more after spinal cord injury, enteric glia transplanted immediately after spinal cord injury survived poorly. This does not impair their ability to be used in patients with spinal cord injury, as most patients are some months or years post-injury.

There was no difference on the speed of migration between the two groups implanted some time after injury. Both migrated at ˜0.3 mm a day in white and gray matter. The cells also migrated through the region of injury. This is important since the enteric glia must migrate some way if they are to create paths for regenerating fibers in the central nervous system. These data therefore further support the potential for use of enteric glia to produce functional recovery after spinal cord injury.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

Barber, P. C.; Neurogenesis and regeneration in the primary olfactory pathway of mammals. Bibl. Anat., 1982, 23: 12-25.
Bartolomei, J. C.; Greer, C. A. Olfactory ensheathing cells: bridging the gap in spinal cord injury. Neurosurgery, 2000, 47: 1057-1069.
Bannerman, P. G. C.; Mirsky, R.; Jessen, K. R. Establishment and properties of separate cultures of enteric neurons and enteric glia. Brain Res., 1988, 440: 99-108.
Beltz, B. S.; Burd, G. D. Peroxidase techniques. In: Immunocytochemical techniques. Cambrige, Mass.: Blackwell Scientific Publications, 1989; 103-111,
Bernstein, C. N.; Vidrich, A. Isolation, identification, and culture of normal mouse colonic glia. Glia, 1994, 12:108-116.
Bligt A. R. Morphometric analysis of a model of spinal cord injury in guinea pigs, with behavioral evidence of delayed secondary pathology. J. Neurol Sci. 1991, 103:156-71.
Bovolenta, P.; Wandosell, F.; Nieto-Sampedro, M. CNS glial scar tissue: a source of molecules which inhibit central neurite outgrowth. Prog. Brain Res., 1992, 94:367-379.
Brittis P. A., and J. G. Flanagan. Nogo domains and a nogo receptor: implications for axon regeneration. Neuron, 2001, 30: 11-14.
Chen, M. S.; Huber, A. B.; Van der Haar, M. E.; Frank, M.; Schnell, L.; Spillmann, A. A.; Schwab, F. C.; Schwab, M. E. Nogo-A is a myelin-associated neurite outgrowth inhibitor and an antigen for nonoclonal antibody IN-1. Nature, 2000, 403:434-439.
Doucette, J. R.; Kiernan, J. A., Flumerfelt, B. A. The re-innervation of olfactory glomeruli following transection of primary olfactory axons in the central or peripheral nervous system. J. Anat., 1983, 137(pt 1):1-19.
Doucette, R.; Diamond, J. The normal and precocious sprouting of heat nociceptors in the skin of adult rats. J. Comp. Neurol., 1987, 261:592-603.
Fawcett, J. W.; Keynes, R. J. Peripheral nerve regeneration. Annu. Rev. Neurosci., 1990, 13:43-60.
Gabella, G. Ultrastructure of the nerve plexuses of the mammalian intestine: the enteric glial cells. Neuroscience, 1981, 6:425-436.
Gershon, M. D. The enteric nervous system: a second brain. Hosp. Practice, 1999, 34:31-42.
Gruner J. A. Yee A. K and Blight A. R. Histological and functional evaluation of experimental spinal cord injury: evidence of a stepwise response to graded compression. Brain Res. 1996; 729:90-101.
Hatten, M. E.; Liem, R. K. H.; Shelanski, M. L.; Mason, C. A. Astroglia in CNS injury. Glia, 1991, 4:223-243.
Horner, P. J.; Gage, F. H. Regenerating the damaged nervous system. Nature, 2000, 407:963-970.
Jaeger, C. B.; Toombs, J. P.; Borgens, R. B. Grafting in acute spinal cord injury: morphological and immunological aspects of transplanted adult rat enteric ganglia. Neuroscience, 1993, 52:333-346.
Jessen, K. R.; Mirksy, R. Astrocyte-like glia in the peripheral nervous system: anti-immunohistochemical study of the enteric glia. J. Neurosci, 1983, 3:2206-2218.
Lawrence, J. M.; Raisman, G.; Mirsky, R.; Jessen, K. R. Transplantation of postnatal rat enteric ganglia into denervated adult rat hippocampus. Neuroscience, 1991, 44:371-379.
Li, Y.; Field, P. M.; Raisman, G. Repair of adult rat corticospinal tract by transplants of olfactory ensheathing cells. Science, 1997, 277:2000-2002.
Mesulam, M. M. Tetramethyl benzidine for horseradish peroxidase neurohistochemistry: a non-carcinogenic blue reaction-product with superior sensitivity for visualizing neural afferents and efferents. J. Histochem. Cytochem., 1978, 26L106-117.
Middlemiss, P. J., Jiang, S., Wang, J. and Rathbone, M. P. A method for purifying enteric glia from rat myenteric plexus. In Vitrol Cellular and Developmental Biology-Animal, 2002, 38:188-190.
Navarro, X.; Valero, A.; Gudino, G., et al. Ensheathing glia transplants promote dorsal root regeneration and spinal reflex restitution after multiple lumbar rhizotomy. Ann. Neurol., 1999, 45:207-215.
Nixon, B. J.; Doucette, R.; Jackson, P.; Diamond, J. Impulse activity evokes precocious sprouting of nociceptive nerves into denervated skin. Somatosens. Res., 1984, 2:97-126.
Pixley, S. K. The olfactory nerve contains two populations of glia, identified both in vivo and in vitro. Glia, 1992, 5:269-284.
Prinjha, R.; Moore, S. E.; Vinson, M.; Blake, S.; Morrow, R.; Christie, G.; Michalovich, D.; Simmons, D. L.; Walsh, F. S. Inhibitor of neurite outgrowth in humans. Nature, 2000, 403:383-384.
Ramon-Cueto, A.; Nieto-Sampedro, M. Regeneration into the spinal cord of transected dorsal root axons is promoted by ensheathing glia transplants. Exp. Neurol., 1994, 127:232-244.
Ramon-Cueto, A.; Plant, G. W.; Avila, J; Bunger, M. B. Long-distance axonal regeneration in the transected adult rat spinal cord is promoted by olfactory ensheathing glia transplants. J. Neurosci., 1998, 18:3803-3815.
Ramon-Cueto, A.; Valverde, F. Olfactory bulb ensheathing glia: a unique cell type with axonal growth-promoting properties. Glia, 1995, 14:163-173.
Ruhl, A.; Trotter, J.; Stremmel, W. Isolation of enteric glia and establishment of transformed enteroglial cell lines from the myenteric plexus of adult rat. Neurogastroenterol. Motil., 2001, 13:95-106.
Saffrey, M. J.; Burnstock, G. Growth of myenteric plexus explant cultures in a serum-free, hormone supplemented culture medium. Int. J. Dev. Neurosci., 1984, 2:591-602.
Schwab M. E. Neurobiology: finding the lost target. Nature, 2000, 403: 257-260.
Tew, E. A. A.; Anderson, P. N.; Saffrey, M. J.; Burnstock, G. Transplantation of the postnatal rat myenteric plexus into the adult rat corpus striatum: an electron microscopic study. Exp. Neurol., 1994, 129:120-129.
Tew, E. A. A.; Saffrey, M. J.; Anderson, P. N.; Burnstock, G. Postnatal rat NADPH-diaphorase-containing myenteric neurons extend processes when transplanted into adult rat corpus striatum. Exp. Neurol., 1993, 124:265-273.
Tew, E. M. M.; Anderson, P. N.; Burnstock, G. Implantation of the myenteric plexus into the corpus striatum of adult rats: survival of the neurons and glia and interactions with host brain. Reparative Neurol. Neurosci., 1992, 4:311-321.
Theriault, E.; Diamond, J. Nociceptive cutaneous stimuli evoke localized contractions in a skeletal muscle. J. Neurophysiol., 1988a, 60:446-4462.
Theriault, E.; Diamond, J. Intrinsic organizations of the rat cutaneous trunci motor nucleus. J. Neurophysiol., 1988b, 60:463-477.
Verdú, E., Garcia-Alias, G., Fores, J., Gudino-Cabrera, G., Nieto-Sampedro, M., and Navarro, X. Effects of ensheathing cells transplanted into photochemically damaged spinal cord. Neuroreport, 2001, 12: 2303-2309.

1. A method of promoting functional regeneration of injured nerve fibres in the nervous system comprising administering an effective amount of an enteric glial cell.

2. The method according to claim 1 wherein the enteric glial cells are Type II enteric glial cells.

3. The method according to claim 1 wherein the enteric glial cells are autologous.

4. The method according to claim 1 for treating a nerve injury.

5. The method according to claim 1 for treating a spinal cord injury.

6. The method according to claim 1 for treating a neurodegenerative disease.

7. The method according to claim 6 wherein the neurodegenerative disease is Alzheimer's disease, Parkinson's disease, multiple sclerosis, Huntington's disease, Bell's palsy, Pick's disease and amyotrophic lateral sclerosis.

8. The method according to claim 1 wherein the functional regeneration is assessed using a behavioural test.

9. The method according to claim 8 wherein the behavioural test is the reflex activation of the cutaneous trunci muscle.

10. A method of inducing the formation of a blood-brain barrier in the nervous system comprising administering an effective amount of an enteric glial cell.

11. The method according to claim 10 wherein the enteric glial cells are Type II enteric glial cells.

12. The method according to claim 10 wherein the enteric glial cells are autologous.

13. The method according to claim 10 for treating a nerve injury.

14. The method according to claim 10 for treating a spinal cord injury.

15. The method according to of claim 10 for treating a neurodegenerative disease.

16. The method according to claim 15 wherein the neurodegenerative disease is Alzheimer's disease, Parkinson's disease, multiple sclerosis, Huntington's disease, Bell's palsy, Pick's disease and amyotrophic lateral sclerosis.
